(19) United States
(12) Patent Application Publication
(10) Pub. No.: US 2026/0251054 A1
(43) Pub. Date: Aug. 27, 2026

(54) Systems and Methods for Determining Properties of a Wellbore

(71) Applicant: Dassault Systemes Americas Corp., Waltham, MA (US)

(72) Inventors: Zhuang Sun, Austin, TX (US); Andrew Fager, Edina, MN (US)

(21) Appl. No.: 19/061,060

(22) Filed: Feb. 24, 2025

(51) Int. Cl. E21B 47/005

(52) U.S. Cl. CPC E21B 47/005; E21B 2200/20

(57) ABSTRACT

Embodiments determine properties of a wellbore. One such embodiment obtains, in a memory, properties of a composite material. A wellbore includes the composite material. For each stage of a plurality of stages of a lifecycle of the wellbore, based on the obtained properties, a respective finite element (FE) model representing the wellbore at the stage is constructed. For each stage of the plurality of stages, a simulation is performed using the respective FE model constructed to determine at least one property of the wellbore at the stage.

### RELATED APPLICATION

This application is related to U.S. Application entitled “Systems and Methods for Determining Properties of a Composite Material” (Attorney Docket No. 4412.1057-000), filed on Feb. 24, 2025. The entire teachings of the above application are incorporated herein by reference.

### BACKGROUND

A number of existing product and simulation systems are offered on the market for the design and simulation of objects, e.g., wellbores. Such systems typically employ computer aided design (CAD) and computer aided engineering (CAE) programs. These systems allow a user to construct, manipulate, simulate, and optimize complex three-dimensional models of objects or assemblies of objects. These CAD and CAE systems provide a model representation of objects, e.g., real-world objects, using edges or lines, in certain cases with faces. Lines, edges, faces, or polygons may be represented in various manners, e.g., non-uniform rational basis-splines (NURBS).

The advent of CAD and CAE systems allows for a wide range of representation possibilities, such as CAD models, for objects. Computer-based models may be programmed in such a way that the model has the properties (e.g., physical, material, or other physics-based) of the underlying real-world object or objects that the model represents. Example properties include stiffness (ratio of force to displacement), plasticity (irreversible strain), and viscosity (resistance to flow of one layer over an adjacent layer), amongst others. When a CAD or other such computer-based model as is known in the art, is programmed in such a way, it may be used to perform simulations of the object that the model represents. For example, a mesh-based model may be used to represent the interior cavity of a vehicle, the acoustic fluid surrounding a structure, or any number of real-world objects. Moreover, CAD and CAE systems, along with computer-based models, can be utilized to simulate engineering systems, such as real-world physical systems, e.g., cars, airplanes, buildings, wellbores, and bridges, amongst other examples. Further, CAE systems can be employed to simulate any variety and combination of behaviors of these physics-based systems, such as noise and vibration.

### SUMMARY

Existing computer-based approaches for wellbore simulation, analysis, and optimization, consider the interplay between various elements and materials in wellbores, such as how pressure from carbon dioxide (CO₂) affects cement.

Problematically, however, existing technologies fail to model effects of chemical reactions, e.g., carbonation reactions, on wellbore material, e.g., cement, mechanical properties and the corresponding impact on large-scale wellbore stability. Therefore, functionality with improved accuracy, performance, and efficacy for determining properties of a wellbore is needed. Embodiments provide such functionality.

An example embodiment is directed to a computer-implemented method for determining properties of a wellbore. The method begins by obtaining, in a memory, properties of a composite material. According to an embodiment, a wellbore includes the composite material. For each stage of a plurality of stages of a lifecycle of the wellbore, based on the obtained properties, the method constructs a respective finite element (FE) model representing the wellbore at the stage. For each stage of the plurality of stages of the lifecycle of the wellbore, the method further performs a simulation using the respective FE model constructed to determine at least one property of the wellbore at the stage.

In an example embodiment, for at least one stage, performing the simulation may include determining one or more effects on the wellbore based on a chemical reaction between the composite material and at least one other material of the wellbore. According to one such embodiment, the chemical reaction may be a carbonation reaction.

In another example embodiment, the obtained properties of the composite material may include indications of respective properties of the composite material at each of the plurality of stages of the lifecycle. According to one such embodiment, the respective properties may include changes in porosity of the composite material. In another such embodiment, the changes in porosity may be based on a carbonation reaction.

According to an example embodiment, the properties may be obtained from a real-world 3D microstructure image of the composite material or a virtual 3D microstructure image of the composite material.

In another example embodiment, the plurality of stages of the lifecycle of the wellbore may include any combination of initial equilibrium, drilling, casing, cement slurry, cement hardening, and injection and reaction.

According to an example embodiment, for a given stage, constructing the respective FE model may include determining homogenized properties of the composite material and constructing the respective FE model based on the determined homogenized properties.

In another example embodiment, for at least one stage, performing the simulation may include performing a fully coupled thermal-hydro-mechanical analysis of the respective FE model.

According to an example embodiment, the determined at least one property may include any combination of stress state, strain state, plastic strain development, and debonding at an interface.

Another example embodiment is directed to a computer-based system for determining properties of a wellbore. The system includes a processor and a memory with computer code instructions stored thereon. The processor and the memory, with the computer code instructions, are configured to cause the system to implement any embodiments or combination of embodiments described herein.

Yet another embodiment is directed to a computer program product for determining properties of a wellbore. The computer program product includes a non-transitory computer-readable medium with computer code instructions stored thereon. The computer code instructions are configured, when executed by a processor, to cause an apparatus associated with the processor to implement any embodiments or combination of embodiments described herein.

It is noted that embodiments of the method, system, and computer program product may be configured to implement any embodiments or combination of embodiments described herein.

An example embodiment for determining properties of a wellbore may perform property homogenization for a composite material, e.g., cement, in a wellbore based on changes in three-dimensional (3D) microstructure of the material due to chemical reactions, e.g., carbonation reactions. Another example embodiment may generate or formulate a multistage wellbore stability model including initial equilibrium, drilling, casing, cement slurry (i.e., cementing), cement hardening, and injection and reaction. Yet another example embodiment may perform a fully coupled thermal-hydro-mechanical analysis, i.e., simultaneously calculating interactions between heat transfer (thermal), fluid flow (hydro), and mechanical deformation, of a multistage wellbore model. An example embodiment may provide or produce a large-scale wellbore model that takes results of a small-scale simulation or analysis as input to the model. Another example embodiment may evaluate an effect of chemical reactions, e.g., carbonation reactions, on wellbore stability including by determining metrics such as stress/strain state, plastic strain development, and debonding at cement-formation and/or cement-casing interfaces, for non-limiting examples.

An example embodiment can evaluate wellbore stability under an effect of cement carbonation reactions. In one such embodiment, the effect of carbonation reactions on cement mechanical properties can be determined via at least one of (1) microstructural simulation, (2) laboratory experiments, and (3) analytical solutions.

Technical improvements realized by embodiments include, for non-limiting examples, providing more accurate simulation results based on a fully coupled model of an entire lifecycle of a wellbore and increased flexibility to incorporate various sources of how carbonation reactions affect cement properties.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 illustrates an example workflow according to an embodiment.

FIG. 2A is an example graph of simulation results of changes in Young's modulus, according to an embodiment.

FIG. 2B is an example graph of simulation results of changes in shear modulus, according to an embodiment.

FIG. 3A is a magnified view of an example casing, cement, and formation near an example wellbore system, according to an embodiment.

FIG. 3B illustrates an example whole simulation domain, according to an embodiment.

FIG. 4 illustrates a multi-stage wellbore FE model, according to an embodiment.

FIG. 5 illustrates example effects of carbonation reactions on wellbore stability, according to an embodiment.

FIG. 6 is a flowchart of a method for determining properties of a wellbore according to an example embodiment.

FIG. 7 is a schematic view of a computer network in which embodiments may be implemented.

FIG. 8 is a block diagram illustrating an example embodiment of a computer node in the computer network of FIG. 7.

### DETAILED DESCRIPTION

A description of example embodiments follows.

Carbon capture, utilization, and storage (CCUS) is a technology to remove CO₂ from the Earth's atmosphere and mitigate global warming. CCUS involves the injection of CO₂ into the subsurface through a wellbore. Cement is a critical component of a wellbore barrier system that provides mechanical stability as well as hydraulic sealing. CO₂ from underground storage can react with hydrated materials in cement (i.e., carbonation reactions). Such reactions have a significant impact on mechanical properties and durability of cement, and therefore wellbore stability. Although many experimental studies have focused on understanding a mechanism of carbonation reactions and their effects on cement, there is no modeling work considering effects of carbonation reactions on cement mechanical properties and the corresponding implications for wellbore stability at a larger scale.

For non-limiting example, an important challenge relating to CCUS systems is maintaining CO₂ in the subsurface for long timespans, e.g., 100 s or 1,000 s of years, or indefinitely. Cement in a CCUS system may be in direct contact with a formation, and if CO₂ leaks through the cement, the CO₂ may infiltrate the formation or other formations, reenter the air, or even find its way into drinking water. If the CO₂ compromises drinking water, this may also be a violation of federal and/or state law in the U.S., such as Environmental Protection Agency (EPA) regulations. A related concern is toxicity in drinking water, for example if CO₂ infiltrating the drinking water also carries with it brine from the subsurface.

While existing solutions in the literature attempt to address the foregoing and other challenges related to wellbore systems, traditional approaches only consider simplistic effects such as pressure from CO₂. In contrast, embodiments are sophisticated and can determine, e.g., the actual effects of carbonation reactions. Embodiments also employ advanced multi-stage models for analyzing properties of a wellbore. For instance, embodiments can divide a wellbore FE model into different stages, e.g., where cement slurry is injected as part of a liquid phase. Embodiments also go beyond the discrete scale to assess or evaluate the phenomenon of CO₂ degradation in a wellbore at a broader scale, e.g., by treating the CO₂ as behaving like a “field” or similar concept. As part of this, embodiments may determine how CO₂ travels or migrates in a wellbore and/or how much of a given cement block is degraded.

Because of these improvements, embodiments can accurately determine wellbore properties. For example, embodiments can accurately determine the properties of a real-world wellbore based on data collected from, e.g., one or more sensors, in the real-world wellbore environment. These determined properties can, in turn, be used to determine if the real-world wellbore is, for instance, structurally stable and, if not, determine and implement fixes to the wellbore to mitigate issues. Fixes to a wellbore may include, for non-limiting examples, squeeze cementing, cement retrofitting, applying a chemical sealant, and applying a casing patch or liner. Responsively, said determined fixes can be applied to the real-world wellbore. Further, embodiments can be used in part of an optimization routine to optimize changes to the real-world wellbore. Likewise, embodiments can be used in an optimization routine to determine an optimized design of a wellbore that is being constructed. In such an embodiment, for example, properties can be obtained from the real-world environment, e.g., via one or more sensors, and the properties can be used in the optimization routine to determine an optimized wellbore design for the real-world environment. Responsively, the wellbore with the optimized design can be constructed in the real-world environment.

It should be noted, however, that embodiments are not limited to determining properties of a wellbore used for, e.g., a CCUS system. For instance, wellbores have a variety of different applications aside from just CCUS technology, such as oil/petroleum and natural gas production. Embodiments are equally useful for these and any other types of wellbore applications.

### Example Workflow

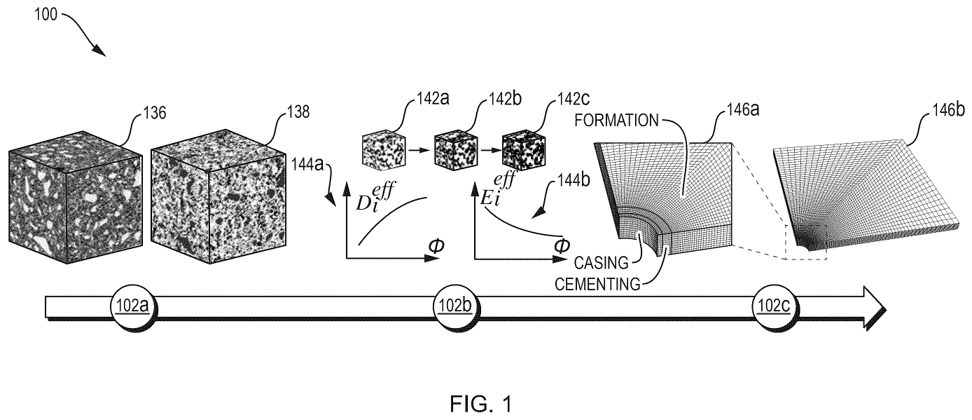

FIG. 1 illustrates an example workflow 100 for determining properties of a wellbore, according to an embodiment. The workflow 100 includes example steps 102a-102c as described hereinbelow.

First, at step 102a, image segmentation may be performed for, e.g., a greyscale 3D microstructure image 136 of cement, to label or otherwise identify individual voxels as belonging to different mineral phases (as shown in image 138 where different color/shading indicates different mineral phases). The result of the image segmentation performed at step 102a is a segmentation of the input image, e.g., the image 136. Example mineral phases according to an embodiment include resolved pores, calcium silicate hydrate (CSH), portlandite (i.e., calcium hydroxide (CH)), and clinker (i.e., an unhydrated phase). In an embodiment, a real-world microstructure image obtained from micro-computed tomography (micro-CT) images in the National Institute of Standards and Technology (NIST) Visible Cement Dataset may be used as an example input. However, it should be noted that other types of known images and/or image sources are also suitable.

Second, at step 102b, image processing may be performed on the segmented image produced at step 102a to mimic chemical reactions, e.g., carbonation reactions including both dry and wet carbonations, which tend to have opposite impacts on cement mechanical properties. The image processing at step 102b may generate new images, e.g., 142a-142c, which indicate changes in mineral phases of the cement caused by the chemical reactions. Simulation steps may be performed for property homogenization and cement mechanical properties may be calculated under various chemical reaction conditions. In other words, the various images produced at step 102b, e.g., 142a-142c, may be used to determine physical properties of the cement in each image 142a-142c. The determined properties can be plotted, as shown by graphs 144a and 144b, to show changes in physical properties of the cement caused by the chemical reactions.

With reference to steps 102a and 102b, a method for generating data from a microstructure simulation may be as described in U.S. Application entitled “Systems and Methods for Determining Properties of a Composite Material” (Attorney Docket No. 4412.1057-000), filed on Feb. 24, 2025, which is herein incorporated by reference in its entirety. While the aforementioned novel method is highly accurate, it should be noted that embodiments are not limited to this method. Any suitable existing method may be used instead. Data from actual physical testing may also be used.

Third, at step 102c, results from the microstructure simulation of step 102b may provide input to a multistage FE model 146a and 146b (where the model 146a is a zoomed in view of the model 146b), which can be used to evaluate wellbore stability under an effect of chemical, e.g., carbonation, reactions. Embodiments'use of homogenized global properties from a microstructure to model a larger scale wellbore 146a and 146b such as at step 102c may also be referred to as a “multiscale” or “upscaling” process. Put another way, embodiments may conduct a continuum-level simulation of a wellbore at a macroscopic level that in turn reflects microscopic variations in different materials that form a composite material such as cement or concrete.

### Example Microstructure Simulation Results

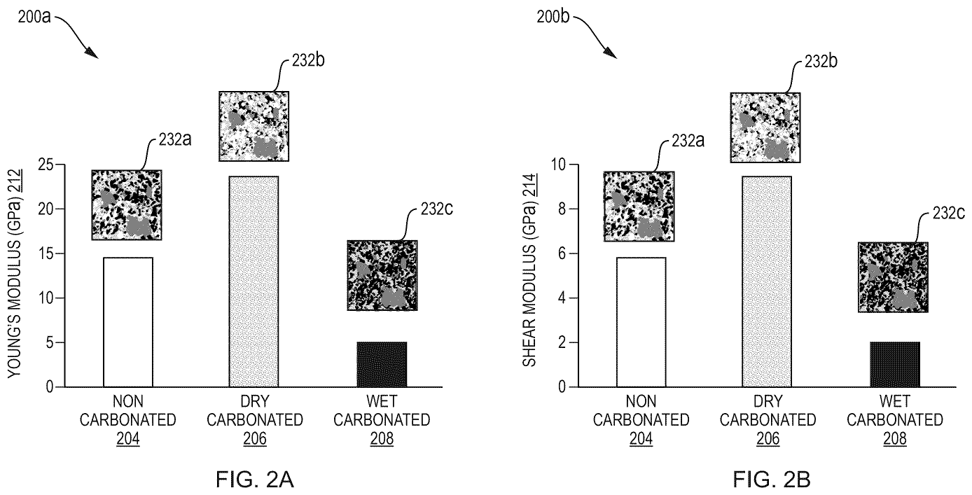

FIG. 2A is an example graph 200a of microstructure simulation results of Young's modulus 212 for noncarbonated 204, dry carbonated 206, and wet carbonated 208 cement, according to an embodiment. As shown in FIG. 2A, corresponding changes in microstructure 232a, 232b, and 232c are also included as references on the top of the columns 204, 206, and 208, respectively. It should be noted that the microstructures 232a-232c are example slices from 3D images for visualization purposes only. To continue, the changes in cement microstructure 232a-232c and the corresponding simulation results of Young's modulus 212 as the carbonation reactions 206 and 208 proceed are consistent with laboratory experimental results (not shown)

FIG. 2B is an example graph 200b of microstructure simulation results of shear modulus 214 for noncarbonated 204, dry carbonated 206, and wet carbonated 208 cement, according to an embodiment. As shown in FIG. 2B, corresponding changes in microstructure 232a, 232b, and 232c are also included as references on the top of the columns 204, 206, and 208, respectively. It should be noted that the microstructures 232a-232c are example slices from 3D images for visualization purposes only. To continue, the changes in cement microstructure 232a-232c and the corresponding microstructure simulation results of shear modulus 214 as the carbonation reactions 206 and 208 proceed are consistent with laboratory experimental results (not shown).

In general, the dry carbonation 206 may tend to generate calcium carbonate (not shown) with a high stiffness, whereas the wet carbonation 208 may tend to dissolve the calcium carbonate. Therefore, the dry carbonation 206 may increase mechanical properties, whereas the wet carbonation 208 may decrease the mechanical properties.

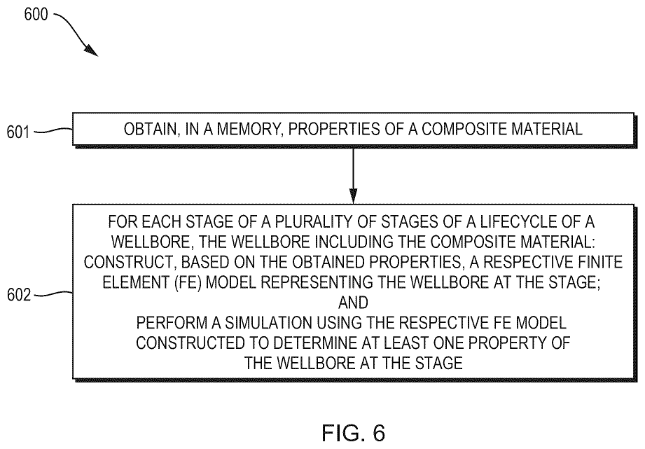

For non-limiting example, the results shown in the graphs 200a and 200b may be determined at step 102b of the workflow 100 described hereinabove in relation to FIG. 1 and at step 602 of the method 600 described hereinbelow in relation to FIG. 6.

### Example Wellbore Simulations

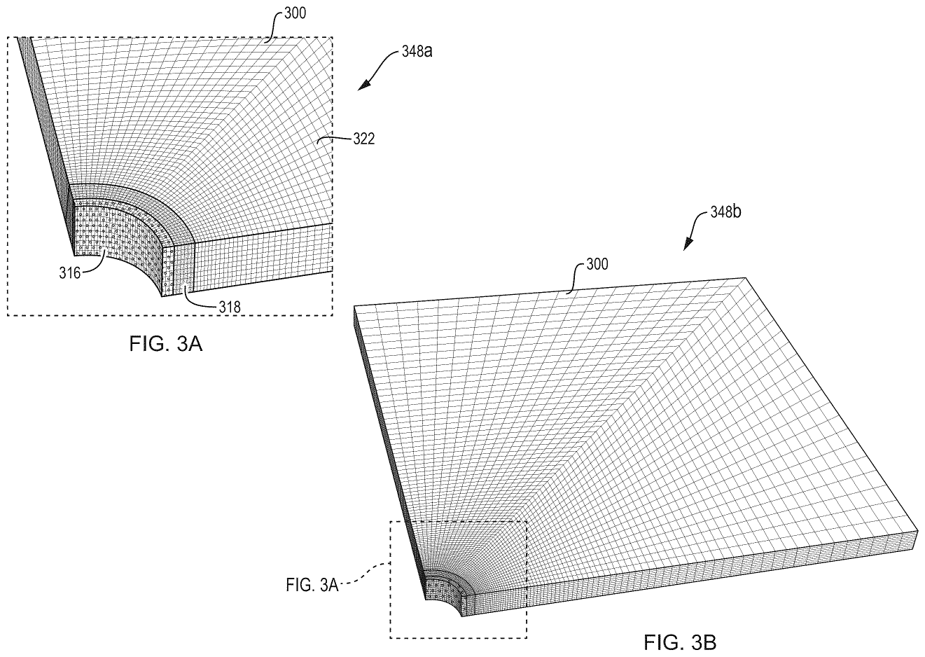

FIG. 3A is a magnified view 348a of an example casing 316, cement 318, and formation 322 of an example wellbore system 300, according to an embodiment. In an embodiment, the casing 316 may be, e.g., steel, while the formation 322 may be, e.g., a porous media such as sandstone.

FIG. 3B illustrates an example whole simulation domain view 348b of the wellbore system 300, according to an embodiment.

According to an embodiment, a stability simulation for the wellbore system 300 may take the microstructure simulation results—described hereinabove with respect to FIGS. 2A and 2B—as input data. In one such embodiment, the stability model for the wellbore system 300 may include the three major components of the casing 316, the cement 318, and the formation 322. An embodiment may only simulate a quarter of the entire wellbore system 300 due to symmetry between the quarter being simulated and the other three quarters of the wellbore system 300. In an embodiment, the bonding of the interface between the cement 318 and the casing 316 and the bonding of the interface between the cement 318 and the formation 322 may also be considered based on cohesive elements with zero initial thickness, i.e., modeling the interfaces using a special type of finite elements that have no initial thickness. For instance, embodiments may simulate bonding of different cohesive elements and/or determine a degree or extent of aperture if debonding occurs. According to another embodiment, a simulation may allow for a fully coupled thermal-hydro-mechanical analysis of the wellbore system 300. Such simulations may be performed at step 102c of the workflow 100 described hereinabove in relation to FIG. 1 and at step 602 of the method 600 described hereinbelow in relation to FIG. 6.

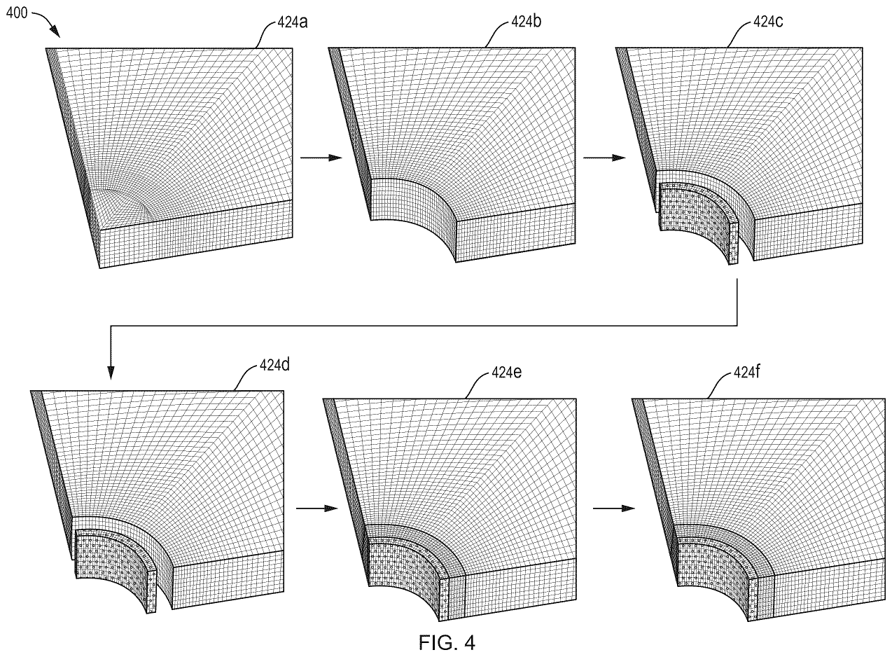

FIG. 4 illustrates a multi-stage wellbore FE model 400, according to an embodiment. In an embodiment, the model 400 includes example model steps of initial equilibrium 424a, drilling 424b, casing 424c, cementing 424d (i.e., cement slurry), cement hardening 424e, and injection and reaction 424f.

In an embodiment, the stages 424a-424f of the model 400 may take place in sequence, and results from simulating one stage may influence simulation(s) of later stage(s). Furthermore, it should be noted that if a given stage, e.g., the injection/reaction stage 424f, is simulated in isolation without accounting for results from previous stage(s), this may lead to decreased accuracy.

Continuing with FIG. 4, simulating wellbore stability subject to chemical reactions, e.g., carbonation reactions, may be a full lifecycle problem that analyzes a complex loading history including, e.g., the initial equilibrium 424a, the drilling 424b, the casing 424c, the cementing 424d, the hardening 424e, and the injection/reaction 424f. Embodiments can generate or formulate a multistage model, e.g., the model 400, which in turn can be used to quantify stress conditions and/or state variables in each well stage, e.g., the stages 424a-424f, and capture an initial stress and/or strain state before modeling a certain stage. In an embodiment, during the injection/reaction stage 424f, carbonation reactions (not shown) between injected CO₂ (not shown) and cement may change the cement mechanical properties and therefore affect wellbore stability.

Continuing again with FIG. 4, in an embodiment, when CO₂ injection takes place during the injection/reaction stage 424f, this may cause rapid change in local pressure and/or temperature near the wellbore (although changes may occur more slowly as distance from the wellbore increases). Thus, at the stage 424f, embodiments may simulate injection in the first instance to generate a stable profile of wellbore pressure and/or temperature. Compared to CO₂ injection, however, carbonation reactions may proceed in a much more gradual fashion. For instance, dry carbonation, e.g., 206 (FIG. 2A), may take place slowly even when large amounts of CO₂ are present. Embodiments can account for the incremental nature of carbonation reactions by simulating this long-term behavior in a so-called “static” step. In an embodiment, a wellbore carbonation reaction that is known to be time-consuming may be simulated by updating mechanical property(ies) of the wellbore cement during a static step in a FE model. Embodiments may employ such a step to replicate effects of a carbonation process, followed by performing a degradation step.

It is noted that simulations performed using the multi-stage wellbore model 400 may be performed at step 102c of the workflow 100 described hereinabove in relation to FIG. 1 and at step 602 of the method 600 described hereinbelow in relation to FIG. 6.

### Example Wellbore Simulation Results

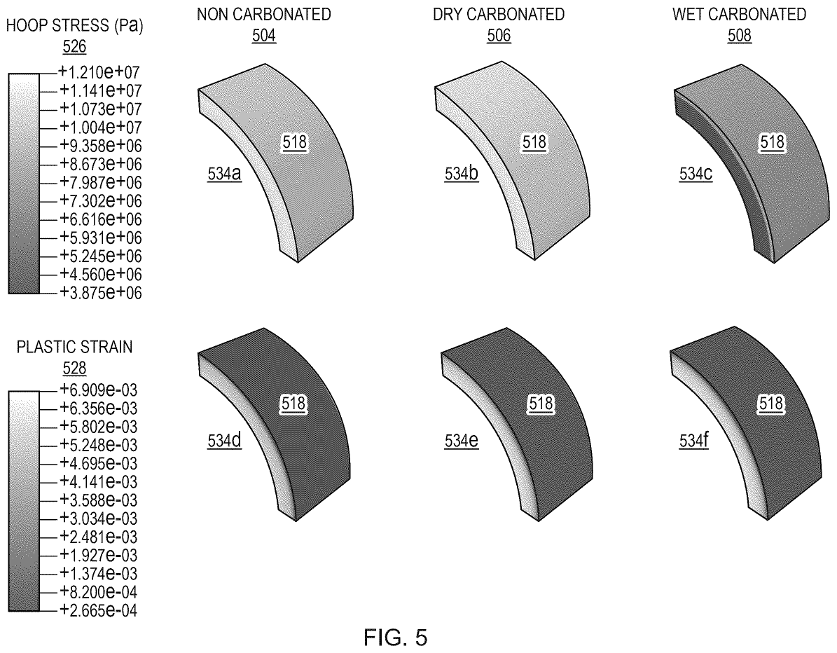

FIG. 5 illustrates example wellbore stability simulation results for simulations 534a-534f as measured by pascals (Pa) of hoop stress 526 and plastic strain 528 for a cement annulus 518 (which may represent, e.g., the cement 318 of FIG. 3A) under noncarbonated 504, dry carbonated 506, and wet carbonated 508 scenarios, according to an embodiment.

An example workflow of embodiments was demonstrated for a use case of the Northern Lights® CCUS project (Øygarden, Norway). The proposed CO₂ storage site is located in the North Sea. The major operator is Equinor® ASA. An embodiment used the characterized stress state and pore pressure from the literature. The injected CO₂ was assigned a temperature of 40° C. cooler than the local formation because actual injection had not started at the time of the demonstration. It should be noted that CO₂ at the Northern Lights project is injected at a depth of approximately 2,000 meters, which generates a considerable amount of pressure.

FIG. 5 shows the simulation results of hoop stress 526 and plastic strain 528 in the cement annulus 518. Specifically, the various shadings of the annulus 518 in the simulations 534a, 534b, and 534c shows the hoop stress 526 for noncarbonated 504, dry carbonated 506, and wet carbonated 508 cement, respectively. Similarly, the various shadings of the annulus 518 in the simulations 534d, 534e, and 534f shows the plastic strain 528 for noncarbonated 504, dry carbonated 506, and wet carbonated 508 cement, respectively. The results depicted in FIG. 5 indicate that thermal contraction caused by the cold injected CO₂ may induce tensile hoop stress 526 and plastic strain 528 in the cement annulus 518. The dry carbonation 506 process may further increase the hoop stress 526 as well as the plastic strain 528. In contrast, the wet carbonation 508 process may mitigate the hoop stress 526 and induce no more plastic strain 528. Therefore, the dry carbonation 506 rather than the wet carbonation 508 can impose an adverse impact on wellbore stability, because the dry carbonation 506 may induce tensile failure and damage to cement, which may later provide a leakage pathway for the injected CO₂.

It is noted that the simulation results shown in FIG. 5 may be determined at step 102c of the workflow 100 described hereinabove in relation to FIG. 1 and at step 602 of the method 600 described hereinbelow in relation to FIG. 6.

### Example Method Embodiment

FIG. 6 is a flowchart of a method 600 for determining properties of a composite material according to an embodiment. The method 600 is computer-implemented and may be implemented using any computing device, e.g., a processor, or combination of computing devices known to those of skill in the art.

The method 600 begins at step 601 by obtaining, in a memory, properties of a composite material, e.g., the cement 318 (FIG. 3A). A wellbore, e.g., the wellbore system 300 (FIG. 3A), includes the composite material. Next, at step 602, for each stage of a plurality of stages of a lifecycle of the wellbore, based on the obtained properties, the method 600 constructs a respective FE model, e.g., the models 424a-424f (FIG. 4), representing the wellbore at the stage. Continuing with step 602, for each stage of the plurality of stages of the lifecycle of the wellbore, the method 600 further performs a simulation using the respective FE model constructed to determine at least one property, e.g., the hoop stress 526 (FIG. 5) or the plastic strain (FIG. 5), of the wellbore at the stage.

To illustrate step 602, consider the example illustrated in FIG. 4 where the wellbore has six stages, namely, initial equilibrium 424a, drilling 424b, casing 424c, cementing 424d, hardening 424e, and injection/reaction 424f. In such an example, at step 602, six models may be generated where each model represents the wellbore during a respective stage. To continue this illustrative example, the six models are in turn used at step 602 to perform simulations where results of the simulations indicate properties of the wellbore at each stage, i.e., initial equilibrium 424a, drilling 424b, casing 424c, cementing 424d, hardening 424e, and injection/reaction 424f.

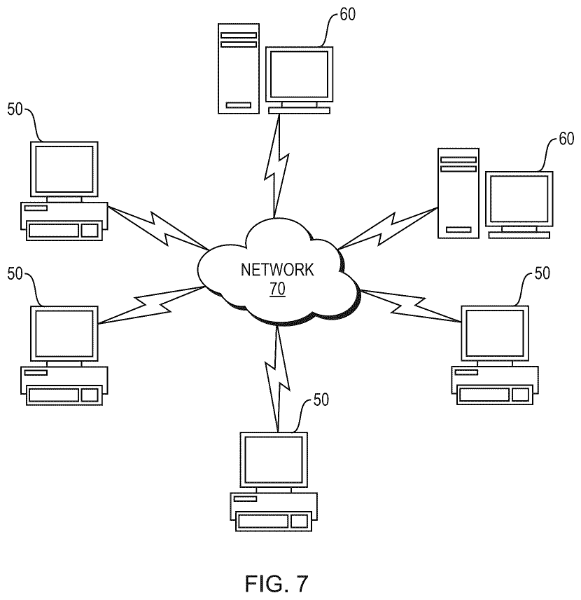

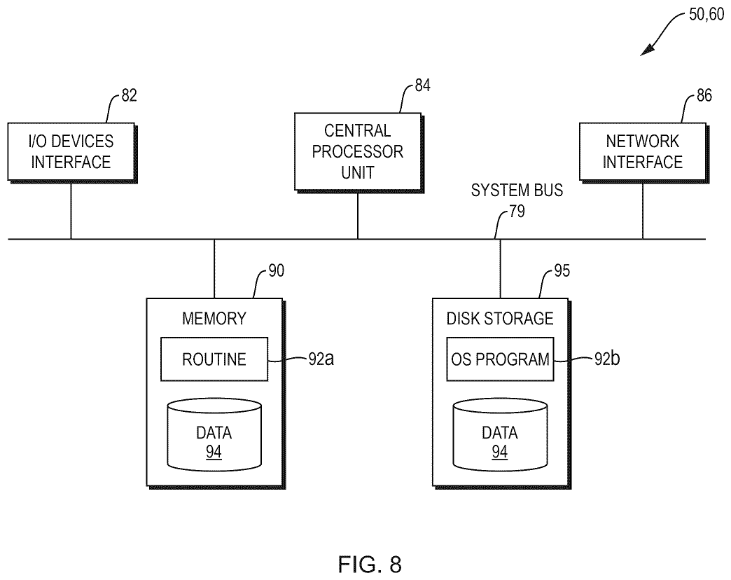

As noted, the method 600 is computer-implemented and, as such, the functionality and effective operations, e.g., the obtaining (601) and constructing and performing (602), are automatically implemented by one or more digital processors. The method 600 can also be implemented using any computing device or combination of computing devices known in the art. Among other examples, the method 600 can be implemented using computer(s)/device(s) 50 and/or 60 described hereinbelow in relation to FIGS. 7 and 8.

In an example embodiment of the method 600, for at least one stage, performing the simulation at step 602 may include determining one or more effects on the wellbore based on a chemical reaction between the composite material and at least one other material, e.g., the casing 316 (FIG. 3A) or the formation 322 (FIG. 3A), of the wellbore. According to one such embodiment of the method 600, the chemical reaction may be a carbonation reaction, e.g., 206 (FIGS. 2A), 208 (FIGS. 2A), 506 (FIG. 5), or 508 (FIG. 5).

In another example embodiment of the method 600, the properties of the composite material obtained at step 601 may include indications of respective properties of the composite material at each of the plurality of stages of the lifecycle. According to one such embodiment of the method 600, the respective properties may include changes in porosity of the composite material. In another such embodiment of the method 600, the changes in porosity may be based on a carbonation reaction.

According to an example embodiment of the method 600, the properties may be obtained at step 601 from a real-world 3D microstructure image, e.g., the image 136 (FIG. 1), of the composite material or a virtual 3D microstructure image of the composite material. In an embodiment of the method 600, at step 601, an image of a microstructure is received and this image is processed, e.g., segmented, at step 601 to determine the properties of the composite material. According to an embodiment, the properties are obtained at step 601 using the functionality performed at steps 102a and 102b of the workflow 100 described hereinabove in relation to FIG. 1.

In another example embodiment of the method 600, the plurality of stages of the lifecycle may include any combination of initial equilibrium (e.g., 424a (FIG. 4)), drilling (e.g., 424b (FIG. 4)), casing (e.g., 424c (FIG. 4)), cement slurry (e.g., 424d (FIG. 4)), cement hardening (e.g., 424e (FIG. 4)), and injection and reaction (e.g., 424f (FIG. 4)).

According to an example embodiment of the method 600, for a given stage, constructing the respective FE model at step 602 may include determining homogenized properties of the composite material and constructing the respective FE model based on the determined homogenized properties.

In another example embodiment of the method 600, for at least one stage, performing the simulation at step 602 may include performing a fully coupled thermal-hydro-mechanical analysis of the respective FE model.

According to an example embodiment of the method 600, the at least one property determined at step 602 may include any combination of stress state (e.g., the hoop stress 526 (FIG. 5)), strain state, plastic strain development (e.g., the plastic strain 528 (FIG. 5)), and debonding at an interface.

Embodiments, e.g., the method 600, can be used as part of a design or development process. For instance, the method 600 can be employed to determine properties of a wellbore used in a setting such as CCUS, oil drilling, or natural gas production, for non-limiting examples. In such an embodiment, based on the determined properties, an existing cement formulation may be adjusted, a different type of cement may be substitute, and/or wellbore design or structure may be modified. Further, embodiments may be utilized in a development process for a real-world composite material to identify potential formulations for the material with different volume fractions of the constituent components of the material.

### Example Advantages

Embodiments can solve the multi-scale challenge of wellbore stability during CO₂ geological storage. Further, embodiments can incorporate how carbonation reactions affect cement mechanical properties in a wellbore stability analysis. Existing wellbore models usually employ a sequential coupling approach of multi-physics behaviors and neglect chemo-mechanical effects. The multistage wellbore stability model of embodiments may use (1) a fully coupled thermal-hydro-mechanical analysis to improve simulation accuracy and/or (2) a static step to simulate long-term mechanical effects of carbonation reactions. In an embodiment, a “static” step may include a computational phase where a wellbore system being modeled is analyzed under equilibrium conditions. A static step may be much faster than a dynamic step because time is not a variable. Moreover, a static step may be suitable for simulating or modeling long-term equilibrium behavior of carbonation reactions.

### Computer Support

Embodiments can be implemented in existing software and CAD and CAE platforms. For instance, embodiments can be implemented using features and functionalities of 3DS SIMULIA® software, including the Abaqus® application by Applicant-Assignee Dassault Systèmes Americas Corporation, among other examples.

FIG. 7 is a schematic view of a computer network in which embodiments may be implemented. Client computer(s)/devices 50 and server computer(s) 60 provide processing, storage, and input/output (I/O) devices executing application programs and the like. Client computer(s)/device(s) 50 can also be linked through communications network 70 to other computing devices, including other client device(s)/processor(s) 50 and server computer(s) 60. The communications network 70 can be part of a remote access network, a global network (e.g., the Internet), cloud computing servers or service, a worldwide collection of computers, local area or wide area networks, and gateways that currently use respective protocols (e.g., TCP/IP, Bluetooth®, etc.) to communicate with one another. Other electronic device/computer network architectures are also suitable.

FIG. 8 is a block diagram illustrating an example embodiment of a computer node (e.g., client processor(s)/device(s) 50 or server computer(s) 60) in the computer network 70 of FIG. 7. Each computer node 50, 60 contains system bus 79, where a bus is a set of hardware lines used for data transfer among components of a computer or processing system. The system bus 79 is essentially a shared conduit that connects different elements of a computer system (e.g., processor, disk storage, memory, I/O ports, network ports, etc.) that enables transfer of information between the elements. Attached to the system bus 79 is an I/O devices interface 82 for connecting various input and output devices (e.g., keyboard, mouse, display(s), printer(s), speaker(s), etc.) to the computer node 50, 60. A network interface 86 allows the computer node to connect to various other devices attached to a network (e.g., the network 70 of FIG. 7). A memory 90 provides volatile storage for computer software instructions 92a and data 94a used to implement an embodiment of the present disclosure (e.g., the method 600 of FIG. 6, etc.). A disk storage 95 provides non-volatile storage for the computer software instructions 92b and data 94b used to implement an embodiment of the present disclosure. A central processor unit 84 is also attached to the system bus 79 and provides for execution of computer instructions.

In one embodiment, the processor routines 92a-92b and data 94a-94b are a computer program product (generally referenced as 92), including a non-transitory, computer readable medium (e.g., a removable storage medium such as DVD-ROM(s), CD-ROM(s), diskette(s), tape(s), etc.) that provides at least a portion of the software instructions for the disclosed system. The computer program product 92 can be installed by any suitable software installation procedure, as is well known in the art. In another embodiment, at least a portion of the software instructions may also be downloaded over a cable, communication, and/or wireless connection. In other embodiments, the disclosure programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present disclosure routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other networks (such as the network 70 of FIG. 7). In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of the computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium, and the like.

In other embodiments, the program product 92 may be implemented as a so-called Software as a Service (SaaS), or other installation or communication supporting end-users.

Embodiments or aspects thereof may be implemented in the form of hardware including but not limited to hardware circuitry, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

For example, the foregoing description and details of embodiments in the figures reference Applicant-Assignee (Dassault Systèmes Americas Corporation) and Dassault Systèmes tools and platforms, for purposes of illustration and not limitation. Other similar tools and platforms are also suitable.

### REFERENCES

Bentz, D. P., Mizell, S., Satterfield, S., Devaney, J., George, W., Ketcham, P., Graham, J., Porterfield, J., Quenard, D., & Vallee, F. (2002). The visible cement data set. Journal of Research of the National Institute of Standards and Technology, 107(2), 137.
Kutchko, B. G., Strazisar, B. R., Lowry, G. V, Dzombak, D. A., & Thaulow, N. (2008). Rate of CO2 Attack on Hydrated Class H Well Cement under Geologic Sequestration Conditions. Environmental Science & Technology, 42(16), 6237-6242.
Li, X.-R., Gu, C.-W., Ding, Z.-C., & Feng, Y.-C. (2023). THM coupled analysis of cement sheath integrity considering well loading history. Petroleum Science, 20(1), 447-459.
Thompson, N., Andrews, J. S., & Bjørnarå, T. I. (2021). Assessing potential thermo-mechanical impacts on caprock due to CO2 injection—a case study from northern lights CCS. Energies, 14(16), 5054.
Walsh, S. D. C., Mason, H. E., Du Frane, W. L., & Carroll, S. A. (2014). Mechanical and hydraulic coupling in cement-caprock interfaces exposed to carbonated brine. International Journal of Greenhouse Gas Control, 25, 109-120.
Zhang, H., Romero Rodriguez, C., Dong, H., Gan, Y., Schlangen, E., & S̆avija, B. (2020). Elucidating the Effect of Accelerated Carbonation on Porosity and Mechanical Properties of Hydrated Portland Cement Paste Using X-Ray Tomography and Advanced Micromechanical Testing. Micromachines, 11(5), 471.
Sun, Z., Fager, A., & Crouse, B. (2024). Modeling of Thermal-Mechanical Impact on Wellbore Integrity Due to CO₂ Injection. ARMA US Rock Mechanics/Geomechanics Symposium. ARMA.

What is claimed is:

1. A computer-implemented method for determining properties of a wellbore, the computer-implemented method comprising, by a processor:
obtaining, in a memory, properties of a composite material; and
for each stage of a plurality of stages of a lifecycle of a wellbore, the wellbore including the composite material:
constructing, based on the obtained properties, a respective finite element (FE) model representing the wellbore at the stage; and
performing a simulation using the respective FE model constructed to determine at least one property of the wellbore at the stage.

2. The computer-implemented method of claim 1, wherein, for at least one stage, performing the simulation includes:
determining one or more effects on the wellbore based on a chemical reaction between the composite material and at least one other material of the wellbore.

3. The computer-implemented method of claim 2, wherein the chemical reaction is a carbonation reaction.

4. The computer-implemented method of claim 1, wherein the obtained properties of the composite material include indications of respective properties of the composite material at each of the plurality of stages of the lifecycle.

5. The computer-implemented method of claim 4, wherein the respective properties include changes in porosity of the composite material.

6. The computer-implemented method of claim 5, wherein the changes in porosity are based on a carbonation reaction.

7. The computer-implemented method of claim 1, wherein the properties are obtained from a three-dimensional (3D) microstructure image of the composite material or a virtual 3D microstructure image of the composite material.

8. The computer-implemented method of claim 1, wherein the plurality of stages of the lifecycle include any combination of:
(i) initial equilibrium, (ii) drilling, (iii) casing, (iv) cement slurry, (v) cement hardening, and (vi) injection and reaction.

9. The computer-implemented method of claim 1, wherein, for a given stage, constructing the respective FE model includes:
determining homogenized properties of the composite material; and
constructing the respective FE model based on the determined homogenized properties.

10. The computer-implemented method of claim 1, wherein, for at least one stage, performing the simulation includes:
performing a fully coupled thermal-hydro-mechanical analysis of the respective FE model.

11. The computer-implemented method of claim 1, wherein the determined at least one property includes any combination of:
(i) stress state, (ii) strain state, (iii) plastic strain development, and (iv) debonding at an interface.

12. A computer-based system for determining properties of a wellbore, the computer-based system comprising:
a processor; and
a memory with computer code instructions stored thereon, the processor and the memory, with the computer code instructions, being configured to cause the computer-based system to:
obtain, in the memory, properties of a composite material; and
for each stage of a plurality of stages of a lifecycle of a wellbore, the wellbore including the composite material:
construct, based on the obtained properties, a respective finite element (FE) model representing the wellbore at the stage; and
perform a simulation using the respective FE model constructed to determine at least one property of the wellbore at the stage.

13. The computer-based system of claim 12, where, for at least one stage, in performing the simulation, the processor and the memory, with the computer code instructions, are configured to cause the computer-based system to:
determine one or more effects on the wellbore based on a chemical reaction between the composite material and at least one other material of the wellbore.

14. The computer-based system of claim 13, wherein the chemical reaction is a carbonation reaction.

15. The computer-based system of claim 12, wherein the obtained properties of the composite material include indications of respective properties of the composite material at each of the plurality of stages of the lifecycle.

16. The computer-based system of claim 15, wherein the respective properties include changes in porosity of the composite material.

17. The computer-based system of claim 16, wherein the changes in porosity are based on a carbonation reaction.

18. The computer-based system of claim 12, where, for a given stage, in constructing the respective FE model, the processor and the memory, with the computer code instructions, are configured to cause the computer-based system to:
determine homogenized properties of the composite material; and
construct the respective FE model based on the determined homogenized properties.

19. The computer-based system of claim 12, where, for at least one stage, in performing the simulation, the processor and the memory, with the computer code instructions, are configured to cause the computer-based system to:
perform a fully coupled thermal-hydro-mechanical analysis of the respective FE model.

20. A computer program product for determining properties of a wellbore, the computer program product comprising a non-transitory computer-readable medium with computer code instructions stored thereon, the computer code instructions being configured, when executed by a processor, to cause an apparatus associated with the processor to:
obtain, in a memory, properties of a composite material; and
for each stage of a plurality of stages of a lifecycle of a wellbore, the wellbore including the composite material:
construct, based on the obtained properties, a respective finite element (FE) model representing the wellbore at the stage; and
perform a simulation using the respective FE model constructed to determine at least one property of the wellbore at the stage.
